(19) United States
(12) Patent Application Publication
(10) Pub. No.: US 2009/0215044 A1
(43) Pub. Date: Aug. 27, 2009

(54) Receptor in Dendritic Cells

(76) Inventor: Gudrun Werner, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/916,909

(22) PCT Filed: Jun. 9, 2006
(86) PCT No.: PCT/EP2006/005561

§ 371 Date: May 8, 2009

(30) Foreign Application Priority Data
Jun. 10, 2005 (GB) 0511844.3

(51) Int. Cl. C12Q 1/68; C07K 14/705; C12Q 1/02; C07J 41/00

(52) U.S. Cl. 435/6; 530/350; 435/29; 552/554

(57) ABSTRACT

GPBAR1 for use as a target for selective inhibition of Th1 mediated immune responses.

The present invention relates to a specific receptor, such as to the G protein-coupled receptor GPBAR1, inter alia expressed in dendritic cells.

The G protein coupled receptor GPBAR1, e.g. disclosed in WO03051923, is a member of the G protein-coupled receptor family. The biological properties of GPBAR1 include regulation of monocyte/macrophage migration/activation, regulation of dendritic cell differentiation/activation, regulation of lymphocyte activation, proliferation and differentiation regulation of inflammation, regulation of cytokine production and/or release, regulation of pro-inflammatory mediator production and/or release, regulation of immune reaction, GLP (glucagon-like peptide)-1 secretion, insulin secretion, appetite, pancreatic regeneration, pancreatic β cell differentiation, pancreatic β cell growth, insulin resistance. Thus, GPBAR1 is indicated to be of interest in relation to methods of treatment of disorders, e.g. including diseases, wherein such biological properties play a causal or contributory role. Such disorders include but are not limited to (chronic) inflammatory diseases, autoimmune diseases, e.g. psoriasis, inflammatory bowel disease, rheumatoid arthritis, systemic lupus erytomatosis or multiple sclerosis, diseases or syndroms in which a significant pathological component is immune suppression, including viral diseases, e.g. AIDS, transplant rejection crisis and other diseases following transplantation, cancer; neurological disorders, such as neurology CNS disorders, cardiovascular disorders.

Psoriasis (vulgaris) is an immune-mediated disease as indicated by the presence of high numbers of dendritic cells, type 1 effector T cells and the corresponding cytokines in lesional skin. The major working hypothesis for immune pathogenesis implies dendritic cells and activated T lymphocytes as central regulators of a complex set of inflammatory reactions in the skin and, directly or indirectly, as the trigger for keratinocyte hyperproliferation (see for instance Chamian F., and Krueger J. G., Curr Opin Rheumatol. 16: 331-337, 2004). Drugs that suppress the exacerbated type 1 inflammatory immune reaction and/or shift the balance towards a Th2 phenotype are expected to represent a new strategy for therapeutic intervention in psoriasis.

It was now surprisingly found that GBPAR1, e.g. upon activation, e.g. upon activation with bile acids, induces the formation of second messengers, cAMP and IP3, and thereby, a cell-type specific biological response program in the target cell. In activated dendritic cells, agonists of GPBAR1 selectively inhibit the production of Th1 cytokines and of TNFα and do not affect the induction of Th2 cytokines. It was also found that, on the other hand, the expression of surface activation markers in dendritic cells is not changed which indicates that the antigen-presenting function of the agonist-treated cells is not affected. Therefore, agonists of GPBAR1 are able to suppress the ongoing Th1-biased inflammatory process, e.g. in psoriasis, e.g. and thereby favorizing the development of Th2 type cells. GPBAR1 is e.g. expected to be useful as a biomarker for dendritic cells specifically monitoring the infiltration of such cells in inflamed skin.

In one aspect the present invention provides
GPBAR1 for use as a target, e.g. as a biomarker in dendritic cells, for selective inhibition of Th1 mediated immune responses;
GPBAR1 for use as a target, e.g. as a biomarker in dendritic cells, for selective inhibition of Th1 cell activation and differentiation: e.g. and
GPBAR1 for use as a target, e.g. as a biomarker in dendritic cells, for favorizing the development of Th2 type cells.

GPBAR1 as used herein includes a polypeptide of the amino acid SEQ ID:NO 2; e.g. encoded by the nucleotide sequence SEQ ID NO:1, and a polypeptide of the amino acid SEQ ID:NO 4, e.g. encoded by the nucleotide sequence SEQ ID NO:3, respectively, and further includes
a polypeptide having at least 95%, 96%, 97%, 98%, or 99% identity to a polypeptide encoded by a polynucleotide of SEQ ID NO.1 or SEQ ID NO:3; and
a polypeptide having at at least 95%, 96%, 97%, 98%, or 99% identity to the polypeptide sequence of SEQ ID NO:2 or SEQ ID NO:4, respectively;
a polypeptide of SEQ ID NO:2 encoded by a sequence other than SEQ ID NO 1, which, as a result of the redundancy (degeneracy) of the genetic code, also encodes the polypeptide of SEQ ID NO:2;
a polypeptide of SEQ ID NO:4 encoded by a sequence other than SEQ ID NO 3, which, as a result of the redundancy (degeneracy) of the genetic code, also encodes the polypeptide of SEQ ID NO:4;
a polypeptide which is encoded by a polynucleotide, preferably with a nucleotide sequence of at least 100 nucleotides, which is obtained by screening a library under stringent hybridization conditions with a labeled probe comprising the sequence of SEQ ID NO: 1 or SEQ ID NO 3 or a fragment thereof, preferably of at least 15 nucleotides;
a polypeptide of SEQ ID NO:2 or SEQ ID NO: 4 encoded by an RNA transcript of an DNA sequence encoding the polypeptide of SEQ ID NO 2 or SEQ ID NO 4; e.g. an RNA transcript of the DNA sequence of SEQ ID NO 1 or SEQ ID NO. 3; or RNA polynucleotides that are complementary thereto;
a polypeptide as described above which is part of a larger protein such as a precursor or a fusion protein,
   e.g. it is often advantageous to include an additional amino acid sequence that contains secretory or leader sequences, pro-sequences, sequences that aid in purification, for instance multiple histidine residues, or an additional sequence for stability during recombinant production and GPBAR1, beside in the form of the “mature” protein may be a part of a larger protein such as a precursor or a fusion protein;
a variant of a polypeptide as described above, e.g. including allelic forms and splice variants; e.g. which variants vary from GBPAR1 by insertions, deletions, and substitutions that may be conservative or non-conservative, or any combination thereof, preferably a splice variant, allelic variant, or polymorphisms, including polynucleotides encoding GBPAR1 having one or more single nucleotide polymorphisms (SNPs), such as variants in which several, for instance from 50 to 30, from 30 to 20, from 20 to 10, from 10 to 5, from 5to 3, from 3 to 2, from 2 to 1 or 1 amino acids are inserted, substituted, or deleted, in any combination;
a fragment of a GPBAR1 comprising an amino acid sequence having at least 30, 50 or 100contiguous amino acids from the amino acid sequence of SEQ ID NO 2 or SEQ ID NO. 4; and a fragment of a GPBAR1 comprising an amino acid sequence having at least 30, 50 or 100 contiguous amino acids truncated or deleted from the amino acid sequence of SEQ ID NO 2 or SEQ ID NO. 4, preferably a fragment that mediates the biological activity of monocyte/macrophage migration/activation, regulation of dendritic cell differentiation, regulation of lymphocyte activation, proliferation and differentiation, regulation of inflammation, regulation of cytokine production and/or release, regulation of pro-inflammatory mediator production and/or release, e.g. biologically similar as GPBAR1, including fragments with a similar activity or an improved activity, or with a decreased undesirable activity compared with GPBAR1, e.g. preferably fragments which contain the epitope of GBPAR1, e.g. preferably fragments which are antigenic or immunogenic in an animal, especially in a human, e.g. which fragments may be e.g. used for screening for agents which selectively inhibit Th1 cell activation, preferably GBPAR 1 includes a polypetide encoded by SEQ ID NO:1 or SEQ ID NO:3, such as a polypeptide of SEQ ID NO:2 or SEQ ID NO:4;
   wherein SEQ ID NO:1, SEQ ID NO:2, SEQ ID NO:3 and SEQ ID NO:4 are as disclosed in WO03051923 (GPBAR1 human nucleotide sequence: SEQ ID NO:1, GPBAR1 human amino acid seqence: SEQ ID:NO 2; GPBAR1 mouse nucleotide sequence: SEQ ID NO:3, GPBAR1mouse amino acid sequence: SEQ ID:NO 4).

The content of WO03051923 is introduced herein by reference.

GPBAR1 may be prepared as appropriate, e.g. according, e.g. analogously, to a method as disclosed in WO03051923.

“The use as a target” as used herein includes the use of GPBAR1, e.g. as a biomarker, e.g. in a method,
for diagnosing disorders which are Th1 cell-driven,
for monitoring the infiltration of dendritic cells in inflamed skin;
for screening for agents which selectively inhibit Th1 cell activation,
for screening for agents which are favorizing the development of Th2 type cells.

In another aspect the present invention provides GPBAR1, e.g. the use of GPBAR1, e.g. the use of GPBAR1 as a biomarker,
for diagnosing disorders which are Th1 cell-driven;
for diagnosing immune disorders which are Th1 cell-driven;
for diagnosing autoimmune disorders which are Th1 cell-driven;
for diagnosing inflammatory disorders associated with (auto)immune disorders which are Th1 cell-driven;
for diagnosing psoriasis, e.g. which is Th1 cell-driven;
for monitoring the infiltration of Th1 cells in inflamed skin, e.g. for monitoring the therapeutic efficacy in the treatment of an individual with an agent which is expected to have an effect on reducing or curing a disorder or disease which is mediated by Th1 cell activation.

Disorders which are Th1 cell-driven include disorders which are mediated by Th1 cell activation and e.g. include immune disorders, such as autoimmune disorders, e.g. (auto)immune disorders associated with inflammation, such as psoriasis, multiple sclerosis, scleoderma, Crohn's disease, e.g. including ulcerative colitis, Behcet's disease, rheumatoid arthritis, autoimmune diabetes, e.g. type 1 diabetes mellitus; or Th-1 cell-driven infectious diseases, e.g. it has been found (see e.g. Mark T. Whary et al, Medicinal Chemistry, Volume 4, No. 5, p. 531-538, 2004) that Th1 -mediated pathology in mouse models of helicobacter disease and diabetes is ameliorated by concurrent anti-inflammatory Th2 responses to parasite antigens and that initial application of these principles is benefiting human patients suffering from helicobacter infection, or it has been found that there is a correlation between the IL-12B gene and Th-1 mediated infectious diseases and it is known that IL-12 expression correlates strongly with Th-1 mediated diseases (see e.g. International Journal of Immunogentics, 33, p. 231-232, 2006).

In another aspect the present invention provides
A method for monitoring the infiltration of dendritic cells in inflamed skin, comprises determining in a sample from inflamed skin originating from an individual suffering from inflamed skin, the level of dendritic cells and comparing said level with the level of Th1 cells determined in a sample from inflamed and non-inflamed skin;
A method for monitoring the therapeutic efficacy in the treatment of an individual with a substance which is expected to have an effect on reducing or curing a disorder or disease which is mediated by Th1 cell activation, which method comprises determining the level of GPBAR1 in a sample of said individual and comparing that level with the level of GPBAR1prior to administration of said substance.

In WO03051923 it is disclosed that a polynucleotide encoding GPBAR1 may be used as a diagnostic kit. Such kit may also be useful for diagnosing Th1 cell-driven disorders, such as psoriasis.

In another aspect, the present invention provides a kit for diagnosing Th1 cell-driven disorders, comprising as a main component
(a) a polynucleotide encoding GPBAR1, preferably of SEQ ID NO:1 or SEQ ID NO:3 of WO03051923, e.g. or a fragment or an RNA transcript thereof;
(b) a nucleotide sequence complementary to that of (a);
(c) GPBAR1, preferably the polypeptide of SEQ ID NO:2 or SEQ ID NO:4 of WO03051923, e.g. or a fragment thereof; or
(d) an antibody to GPBAR1, preferably an antibody to GPBAR1 of SEQ ID NO:2 or SEQ ID NO:4 of WO03051923.

In WO03051923 is disclosed that GPBAR1 may be used for the screening of compounds to identify compounds that stimulate or inhibit the function or level of the polypeptide. Such methods may identify agonists which are expected to be useful for the treatment of Th1 cell-driven disorders, e.g. psoriasis.

In another aspect, the present invention provides a kit for the screening to identify compounds that mediate the function or level of GPBAR1 for use in the treatment of Th1 cell-driven disorders, e.g. psoriasis, comprising as a main component
(i) GPBAR1,
(ii) cells or membranes expressing GPBAR1, or
(iii) a fusion protein comprising GPBAR1.

A kit according to the present invention, beside (a), (b), (c) or (d); or, (i), (ii), or (iii), respectively, may comprise a substantial component, e.g. including an appropriate environment of a sample to be tested, e.g. and appropriate means to determine the effect of any of a), b), c) or d) in a sample to be tested.

In another aspect the present invention provides a kit for diagnosing of a disorder or disease which is mediated by Th1 cell activation in a sample of an individual, or a kit for diagnosing Th1 cell-driven disorders, comprising
A) a molecule which recognizes GPBAR1, optionally in a labeled form,
B) instructions for use,
C) optionally detection means, and
D) optionally a solid phase.

In another aspect the present invention provides a method for diagnosing a disorder or disease which is mediated by Th1 cell activation, comprising
a) providing a sample of an individual,
b) determining the level of GPBAR1 in said sample,
c) comparing the level of GPBAR1 as determined in step b) with a reference level from a sample of a healthy control individual, and
d) diagnosing a disorder or disease which is mediated by Th1 cell activation by determination whether the level of GPBAR1 as determined in step b) is different from said reference level.

In another aspect the present invention provides a method for identifying an agent that modulates Th1 cell activation, e.g. and using such agent, comprising
a) determining the level of GPBAR1 in a GPBAR1 specific test system, in the absence and in the presence of a candidate compound which is expected to modulate the level of GPBAR1,
b) identifying a candidate compound which modulates the level of GPBAR1 as determined in step a) as an agent;

e.g. and using an agent identified is used as a pharmaceutically active agent, e.g. for the preparation of a medicament, in the treatment of disorders or diseases mediated by Th1 cell activation.

In another aspect the present invention provides a method for screening to identify compounds that mediate the function or level of GPBAR1 for use in the treatment of Th1 cell-driven disorders, e.g. psoriasis, comprising
(a) measuring or, detecting, quantitatively or qualitatively, the binding of a candidate compound to
   (i) GPBAR1,
   (ii) the cells or membranes expressing GPBAR1, or
   (iii) to a fusion protein comprising GPBAR1, by means of a label directly or indirectly associated with the candidate compound;
(b) measuring the competition of binding of a candidate compound to any of (i), (ii) or (iii) as defined in (a) in the presence of a labeled competitor;
(c) determining whether the candidate compound results in a signal generated by activation of GPBAR1, using detection systems appropriate to the cells or cell membranes expressing GPBAR1,
(d) mixing a candidate compound with a solution containing a GPBAR1, to form a mixture, measuring activity of said GPBAR1 in the mixture, and comparing the activity of the mixture to a control mixture which contains no candidate compound; and
(e) detecting the effect of a candidate compound on the production of mRNA encoding any of (i), (ii) or (iii) as defined in (a), e.g. by use of an ELISA assay,
   and choosing a candidate compound from which an agonistic effect in any of (a), (b), (c), (d) or (e) has been detected;
   e.g. and using such candidate compound chosen for the treatment, or for the preparation of a medicament for the treatment, of Th1 cell-driven disorders, e.g. psoriasis.

A candidate compound includes compound (libraries), from which the effect on any of (a), (b), (c), (d) or (e) is unknown. Examples of potential agonists according to the present invention include compounds which bind to a polypeptide according to the present invention, e.g. including oligopeptides, polypeptides, proteins, mimetics, small molecules, e.g. low molecular weight compounds (LMW's), preferably LMW's.

An agent as used herein is a candidate compound from which pharmaceutical activity, e.g. GPBAR1 agonistic activity, has been detected in a method provided by the present invention.

Determination of the level (amount) of GPBAR1 may be carried out as appropriate, e.g. according, e.g. analogously, to a method as conventional or as described herein. Any method provided by the present invention may be carried out as appropriate, e.g. according, e.g. analogously, to a method as conventional or as described herein.

In another aspect the present invention provides the use of an agent, e.g. agonist of GPBAR1, e.g. in the form of a pharmaceutical composition, for the preparation of a medicament for the treatment of Th1 cell-driven disorders, e.g. psoriasis.

In another aspect the present invention provides a pharmacutical composition comprising an agonist of GPBAR1 together with pharmaceutically acceptable excipient, e.g. for use in the treatment of Th1 cell-driven disorders.

In another aspect the present invention provides a method of treating Th1 cell-driven disorders, e.g. psoriasis, comprising administering an effective amount of an agent, such as an agonist of GPBAR1, to a subject in need of such treatment.

Disorders as used herein include diseases.

We have also found compounds which are agonists of GPBAR1, e.g. as is evident from the Examples and Figures below.

In another aspect the present invention provides the use of an agonist of GPBAR1 which is selected from bile acids, e.g. selected from taurolithocholic acid, taurodeoxycholic acid, taurochenodeoxycholic acid and taurocholic acid, as a reference substance in a use, a screening method or a kit provided according to the present invention.

In another aspect the present invention provides the use of an isolated polypeptide selected from one of the groups consisting of
a) an isolated polypeptide encoded by a polynucleotide of SEQ ID NO.1 or SEQ ID NO:3 of WO03051923;
b) an isolated polypeptide having at least 95%, 96%, 97%, 98%, or 99% identity to a polypeptide encoded by a polynucleotide of SEQ ID NO.1 or SEQ ID NO:3 of WO03051923,
c) an isolated polypeptide of SEQ ID NO:2 or SEQ ID NO:4 of WO03051923,
d) an isolated polypeptide having at at least 95%, 96%, 97%, 98%, or 99% identity to the polypeptide sequence of SEQ ID NO:2 or SEQ ID NO:4 of WO03051923, and optionally
e) fragments and variants of polypeptides as defined in a), b), c) or d), for the screening of compounds which are useful in the treatment of psoriasis.

### DESCRIPTION OF THE FIGURES

FIG. 1

### Calcium Mobilization by Bile Acids in NT50 Cells (See Example 1A)

Shows calcium mobilisation by a receptor of SEQ ID:NO 2 of WO03051923 after stimulation with bile acids. Measured on a 96 well FLIPR instrument. Fluorescence signals are represented as percent of responses obtained by 2.5 μM TLCA in control wells. TLCA (taurolithocholic acid, —); TDCA (taurodeoxycholic acid, ▪—▪); TCDCA (taurochenodeoxycholic acid, ♦—♦); TCA (taurocholic acid, ▴—▴)

FIG. 2

### Receptor Desensitization by Bile Acids in NT50 Cells (See Example 1a))

Shows the specificity of calcium responses triggered by bile acids by GPBAR1 of SEQ ID:NO 2 of WO03051923 after activation and subsequent receptor desensitization. Tested in a second stimulation by a saturating amount of TLCA (2.5 μM). Maximal signals obtained for a particular well are represented as percent of controls which are pre-treated with assay buffer alone. Compounds are tested in duplicates at 8 concentrations TLCA (taurolithocholic acid, —); TDCA (taurodeoxycholic acid, ▪—▪); TCDCA (taurochenodeoxycholic acid, ♦—♦); TCA (taurocholic acid, ▴—▴)

FIG. 3

TLCA Induced Increases Of cAMP Levels (See Also Example 1b))

Jurkat NT51 host cells, Jurkat NT50 and HEK-LFR6.1 cells GBPAR1 of SEQ ID NO:2 of WO03051923 are tested with TLCA at increasing concentrations for intracellular cAMP at 30 minutes after compound addition. Data are expressed as normalised fluorescence values and are the mean (+/−sd) of 4 replicates.

FIG. 4

Cytokine Production by LPS-Activated Dendritic Cells Treated with Agonists of a Receptor of GPBAR1 of SEQ ID NO:2 of WO03051923

Dendritic cells are generated from human monocytes by treatment with GM-CSF and IL-4and TLCA (10 μM) is added to the cultures on day 0 or day 6, the timepoint of stimulation by LPS. Cytokine levels in culture supernatants are analysed at 48 hrs after activation. Data are represented as percent of vehicle treated controls calculated from duplicate determinations.

FIG. 5

Dose Dependent Inhibition of Th1 Cytokine Production in Dendritic Cells Treated with TLCA

Dendritic cells are generated from human monocytes by treatment with GM-CSF and IL-4and TLCA is added to the cultures on day 0. On day 6 cells are stimulated with LPS in the presence of varying amounts of TLCA. Cytokine levels in culture supernatants are analysed at 48 hours after activation. Data are presented as percent of vehicle treated (DMSO) controls.

FIG. 6

### Surface Activation Markers of Dendritic Cells Treated With TLCA

Dendrite cells are stained for expression of surface markers at 48 hours after activation by LPS and 10 μM TLCA or LPS and the corresponding amount of vehicle (DMSO) and analysed by FACS. Histograms represent:

Gray, filled: DC treated with LPS; dark, unfilled: DC not treated with LPS; light, unfilled: isotype controls

Treatment of dendritic cells with an agonist of a receptor of SEQ ID:NO 2 does not affect the expression of surface molecules known to be involved in the antigen presenting function.

This indicates that the development of certain T cell responses such as for instance Th2 cells are reasonably expected to proceed normally.

FIG. 7

### Cytokine Production in Co-Coltures of GPBAR1 Agonist Treated Dendritic Cells and Allogeneic CD4⁺ T Cells.

Monocyte-derived dendritic cells activated by LPS (mature dendritic cells) on day 6 after plating or left unstimulated (immature dendritic cells) are co-cultured with allogeneic CD4⁺ T cells for 72 hours in the presence of 10 μM TLCA. Cytokine levels in supernatants are represented as percent of vehicle (DMSO) treated controls. Data are the mean of duplicate wells.

Since IFNγ production is drastically inhibited while IL-4 is not it is concluded that the development of Th1 cells from naive T cells encountering dendritic cells that are triggered by PBAR1 agonists will be markedly restrained in favor of Th2 cells.

In the following Examples all tempratures are in ° Celsius.

The Following abbreviations are used:
GPBAR1 the receptor of SEQ ID NO:2 of WO03051923
TLCA taurolithocholic acid
TDCA taurodeoxycholic acid
TCDCA taurochenodeoxycholic acid
TCA taurocholic acid

### EXAMPLE 1

Calcium and cAMP Functional Assays with Agonists of GPBAR1

### A) Calcium Assay

Jurkat cells expressing recombinant GPBAR1 (NT50 cells) have been shown to be coupled functionally to activation of PLC and calcium mobilization, and to cAMP formation. Basal calcium levels in GPBAR1- or vector control-transfected Jurkat cells are observed to be in the normal, 100 nM to 200 nM, range. Agonistic triggering of a GPBAR1 in stable transfectants induced transient rises in cytoplasmatic calcium levels while the same compounds do not change calcium levels in control cells.

Jurkat NT50 cells are loaded with Fluo-4 AM and various steroidal substances (including bile acids) are evaluated for agonist induced calcium mobilization on a FLIPR instrument (Molecular Devices, Sunnyvale, USA). Briefly, NT50 cells are harvested from tissue culture flasks by centrifugation at 200 g and re-suspended in Fluo-4 AM dye loading solution (3 ml per 1×10⁷ cells containing 1 μM Fluo-4 AM, 2 mM probenecid in complete RPMI medium buffered with 20 mM HEPES, pH7.4) on the day of the experiment. After incubation for 1hour at 37° cells are washed twice (centrifugation at 200 g) with 10 ml assay buffer (5.4 mM KCl, 0.8 mM MgSO₄, 120 mM NaCl, 10 mM D-Glucose, 20 mM HEPES (pH 7.4) and 2 mM CaCl₂) containing 2 mM probenecid. Cells are then counted and transferred into black/clear bottom 96-well plates (Corning Inc., New York, USA) at 2×10⁵ cells per well in 75 μl assay buffer. Assay plates are centrifuged for 2 min at 200 g before use.

Serial dilutions of test substances (10 mM stock in DMSO) are performed in assay buffer containing 20% DMSO. Pre-diluted samples are then transferred into 96 well compound plates and supplemented with assay buffer to obtain 4× the test concentrations (6 μl compound dilution + 194 μl buffer). Aliquots of 25 μl are added onto the cells using the FLIPR 96-tip pipette and changes in fluorescence intensities are recorded at an interval of one second over a period of 2 minutes.

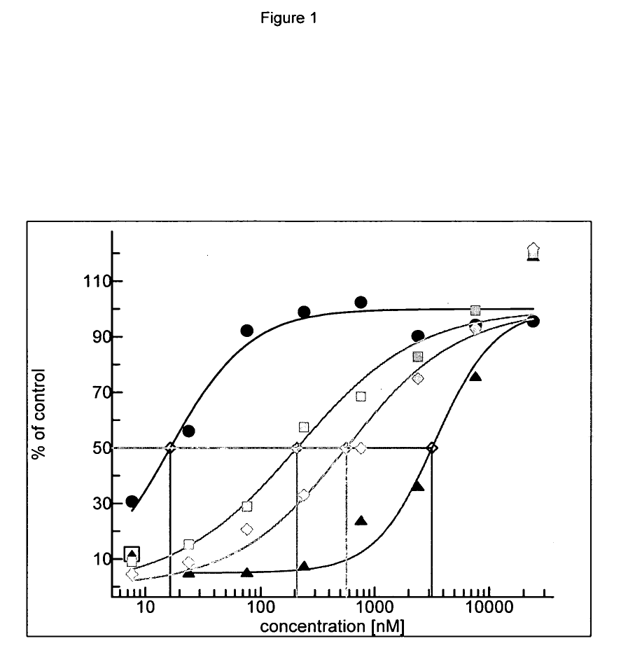

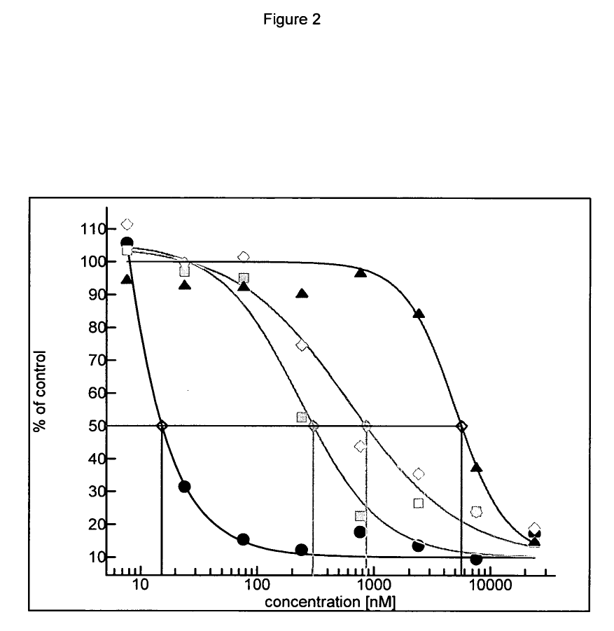

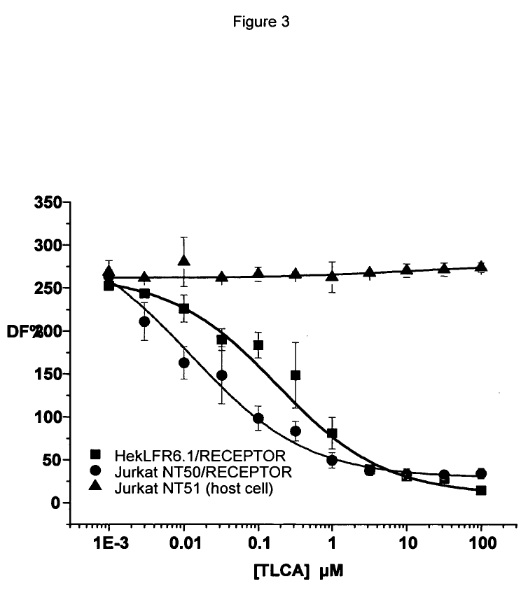

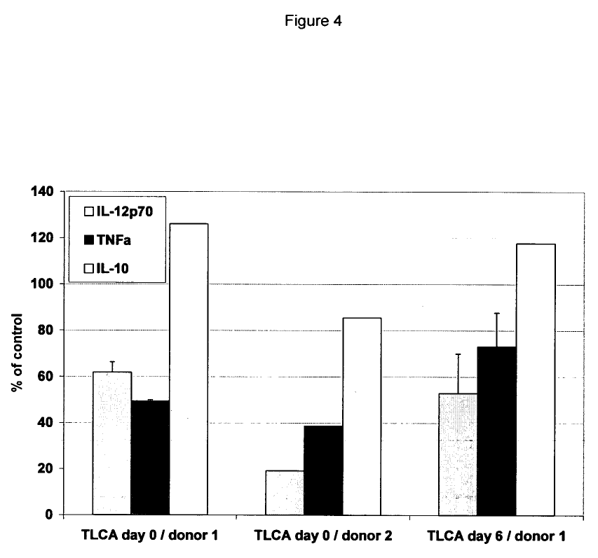

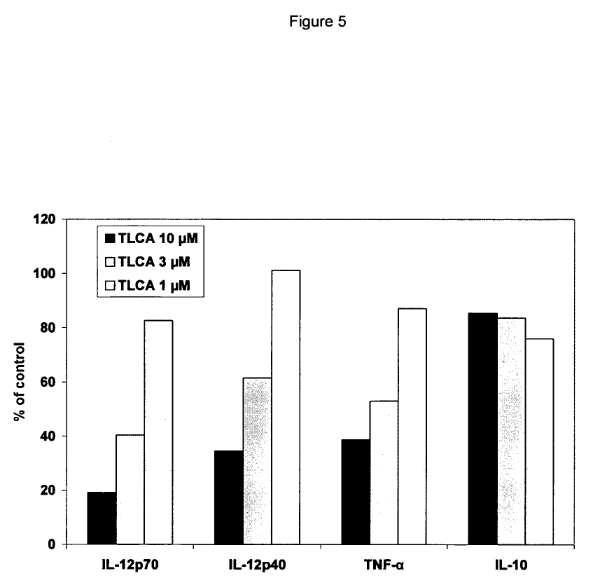

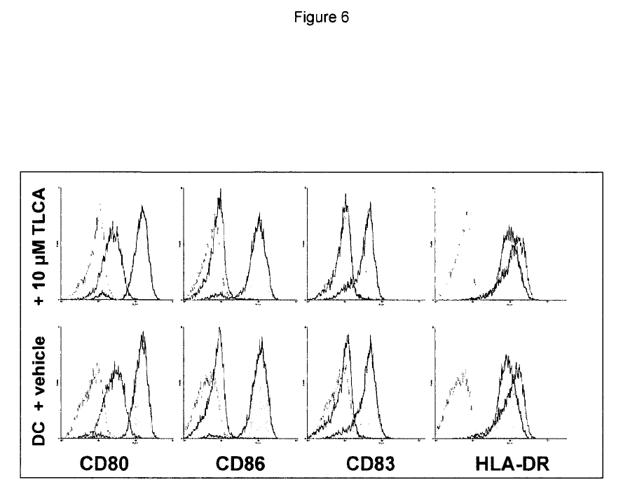

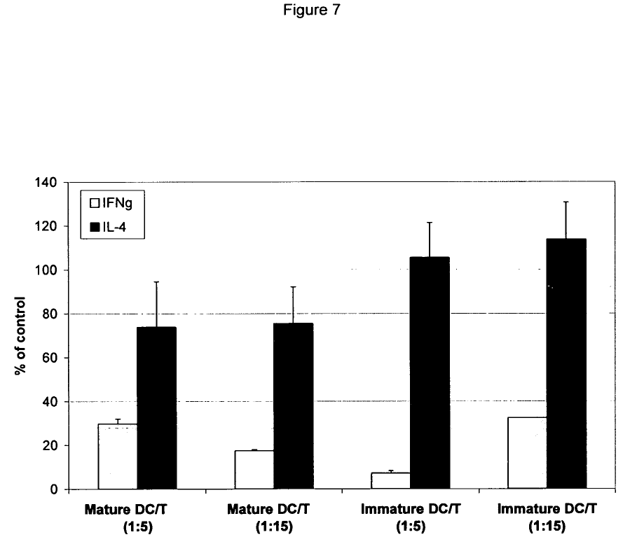

Of more than 200 steroidal compounds tested taurolithocholic acid (TLCA) is the most potent agonist (EC₅₀=70.9±13.1 nM). Data of one representative experiment with different bile acids are shown in FIG. 1. Jurkat control cells transfected with the vector alone do not mobilize calcium by bile acids. The specificity of calcium responses triggered by bile acids through GPBAR1 is indicated further by GPBAR1 desensitization after activation (see FIG. 2) tested in a second stimulation by a saturating amount of TLCA (2.5 μM).

B) cAMP Assay

Experiments to determine changes in cAMP after addition of steroidal compounds to Jurkat NT50, NT51 control cells and of HEK-LFR cells expressing a GPBAR1 are performed with the HTRF kit from CIS Bio International (Bagnols sur Ceze, France) according to instructions of the manufacturer. The method is based on a competitive immunoassay between native cAMP produced by cells and added cAMP labeled with XL665. Binding of the tracer to an anti-cAMP monoclonal antibody labeled with an Europium cryptate. is detected by fluorescence energy transfer (FRET). The specific signal (HTRF, time-resolved fluorescence) is inversely proportional to the concentration of native cAMP in the sample. Data are calculated from intensities of emitted light filtered at two wavelengths L1 (665 nM) and L2 (620 nM) as the ratio L1/L2 and normalised by

ΔF=[(sample ratio−negative ratio)/negative ratio]×100

Data of a typical experiment are set out in FIG. 3.

### EXAMPLE 2

Cytokine production in dendritic cells treated with a GPBAR1 agonists

Dendritic cells are generated in-vitro from human monocytes by culturing in media containing GM-CSF and IL-4 as described (Bender, A., Sapp, M., et al. J. Immunol. Methods 196, 121-135 (1996)). Briefly, leukocyte concentrates are prepared from buffy coats using Lymphoprep™ (Technoclone, Vienna, Austria) according to manufacturer's instructions. Monocytes are isolated using a magnetic beads monocyte isolation kit (Miltenyi Biotec GmbH, Bergisch Gladbach, Germany). The purity of monocyte preparations is analysed by FACS staining of CD14 positive cells using fluorescently labeled antibodies (BD Biosciences, New York, USA) and is generally >95%. To differentiate into dendritic cells monocytes are resuspended at a density of 0.5×10⁶ in RPMI medium containing antibiotica (1% penicillin, 1% streptomycin), 2 mM L-glutamine, 10% FCS, human GM-CSF (100 ng/ml) and IL-4 (80ng/ml), and test compounds at various concentrations, and are seeded into 6 well Costar cell culture plates (Corning Inc., New York, USA) at 5 ml per well Cells are incubated at 37° in a humidified chamber containing 5% CO₂. On day 3 after plating cells are fed by replacing 2.5ml of supernatant with fresh medium. On day 6 cells are stimulated with LPS (Sigma, Taufkirchen, Germany) at 100 ng/ml by replacing 1 ml supernatants with 5-fold the amount of LPS together with test compounds in fresh culture medium. After 48 hours of incubation supernatants are analysed for cytokine contents using the BD™ Cytometric Bead Array kit (BD Biosciences, New York, USA) simultaneously detecting IL-8, IL-1b, IL-6, IL-10, TNF-a, and IL-12p70. IL-12p40 in cell culture supernatants is determined by ELISA (BD Biosciences, New York, USA). IL-12 is known to be a key cytokine driving Th1-mediated autoimmune diseases, whereas e.g. IL-10 is known to be a key cytokine driving Th2-mediated autoimmune diseases.

As shown in FIG. 4 and FIG. 5 treatment of dendritic cells during differentiation from monocytes by TLCA specifically and dose-dependently inhibits the production of IL-12p70 and TNF-α after activation by LPS. The production of IL-10 and other cytokines is not affected by TLCA. Similar results are obtained when TLCA is added to dendritic cells on day 6 together with the LPS stimulus (see FIG. 4).

Similarly to IL-12p70 the production of IL-12p40 which in addition to IL-12 is also a component of biologically active IL-23 is dose dependently inhibited by TLCA in dendritic cells activated by LPS (see FIG. 5). Therefore, the de-novo formation of Th1 cells from naive T cells as well as the activation of memory T cells is affected upon encounter with dendritic cells which have been confronted with GPBAR1 agonists.

### EXAMPLE 3

Activation Markers in Dendritic Cells Treated with Gpbar1 Agonists

Dendritic cells are generated in-vitro from human monocytes as described in example 2. On day 6 after plating cells are activated by LPS and varying amounts of GPBAR1 agonist or vehicle or left untreated and 48 hours later analysed for expression of surface activation markers by FACS. Staining of cells is performed using fluorescently labeled monoclonal antibodies from BD Biosciences (New York, USA) according to standard protocols provided by the manufacturer. A typical staining profile is shown in FIG. 6.

### EXAMPLE 4

Cytokine Production in Co-Cultures of T Cells and Dendritic Cells Treated with GPBAR1 Agonists

Dendritic cells are cultured as described in example 2 and stimulated by 100 ng/ml on day 6or left unstimulated for 48 hours and GPBAR1 agonists or the corresponding vehicle controls are provided together with the stimulus. Two days later, purified, allogeneic CD4⁺ T cells (MACS CD4⁺ T cell isolation kit from Miltenyi Biotec GmbH, Bergisch Gladbach, Germany ) are added at ratios 5:1 and 15:1 to mature (LPS-treated) or immature (un-stimulated) DC. Briefly, Dendritic cells of day 8 are re-plated into 96 well U-bottom cell culture plates (Corning Inc., New York, USA) at 2.5×10⁴ cells per well or at 8.3×10⁴ in 100 μl medium and 1.3×10⁵ CD4+ T cells in 100 μl medium are added to each well. Co-cultures are incubated for 72hours at 37° in a humidified chamber containing 5% CO₂. Th1/Th2 cytokine levels in supernatants are then analysed using the BD™ Cytometric Bead Array kit (BD Biosciences, New York, USA). Results of one representative experiment are shown in FIG. 7.

1. GPSAR1 for use as a target for selective inhibition of Th1 mediated immune responses,

2. GPBAR1 for use as a target for selective inhibition of Th1 cell activation,

3. The use of GPBAR1 for diagnosing disorders which are Th1 cell-driven,

4. The use according to claim 3 for diagnosing psoriasis.

5. GPBAR1 as a biomarker in dendritic ceils for use according to claim 1.

6. Kit for diagnosing Th1 cell-driven disorders, comprising as a main component
(a) a polynucleotide encoding GPBAR1, or a fragment or an RNA transcript thereof;
(b) a nucleotide sequence complementary to that of (a);
(c) GPBAR1, or
(d) an antibody to GPBAR1.

7. Kit for the screening to identify compounds that mediate the function or level of GPBAR1 for use in the treatment of Th1 cell-driven disorders, comprising as a main component
(i) GPBAR1,
(ii) cells or membranes expressing GPBAR1, or
(iii) a fusion protein comprising GPBAR1,

8. A method for diagnosing a disorder or disease which is mediated by Th1 cell activation, comprising
a) providing a sample of an individual,
b) determining the level of GPBAR1 in said sample,
c) comparing the level of GPBAR1 as determined in step b) with a reference level from a sample of a healthy control individual, and
d) diagnosing a disorder or disease which is mediated by Th1 cell activation by determination whether the level of GPBAR1 as determined in step h) is different from said reference level

9. A method for monitoring the therapeutic efficacy in the treatment of an individual with an agent which is expected to have an effect on reducing or curing a disorder or disease which is mediated by Th1 cell activation, which method comprises determining the level of GPBAR1 in a sample of said individual and comparing that level with the level of GPBAR1 prior to administration of said agent.

10. A method for identifying an agent that modulates Th1 cell activation comprising
a) determining the level of GPBAR1 in a GPBAR1 specific test system, in the absence and m the presence of a candidate compound which is expected to modulate the level of GPBAR1,
b) identifying a candidate compound which modulates the level of GPBAR1 as determined in step a) as an agent.

11. A method according to claim 10, wherein an agent identified is used as a pharmaceutically active agent in the treatment of disorders or diseases mediated by Th1 cell activation.

12. The use of an agonist of GPBAR1 which is selected from taurolithocolic acid, taurodeoxycholic acid, taurochenodeoxycholic acid and taurocholic acid, as a reference substance in a use, a screening method or a kit according to claim 1.
